(19) United States
(12) Patent Application Publication
(10) Pub. No.: US 2005/0272116 A1
(43) Pub. Date: Dec. 8, 2005

(54) Polymer substrate for recombinant protein purification and environmental remediation

(75) Inventors: Michael S. Ellison, Pickens, SC (US); Albert Abbott, Clemson, SC (US); Darren Baker, Westminister, SC (US)

Correspondence Address: DORITY & MANNING, P.A., POST OFFICE BOX 1449, GREENVILLE, SC, 29602-1449, US

(73) Assignee: Clemson University

(21) Appl. No.: 10/861,660

(22) Filed: Jun. 4, 2004

(51) Int. Cl. C07K 1/10; C12P 21/06

(52) U.S. Cl. 435/69.1; 530/416

(57) ABSTRACT

A polymer substrate for recombinant protein purification is provided. A process for purifying recombinant proteins is also provided. In general, the present invention is directed to a chelating composition which may be used in a self-supported purification medium for recombinant protein purification. In another embodiment, a process for removal of transition metals from environmental waste is also provided.

### BACKGROUND

While recombinant protein development is vital to areas in the biochemical field, an efficient and effective method for recombinant protein purification remains a major obstacle. Existing methods typically involve metal ion affinity chromatography; these methods, however, have many limitations.

Such existing methods of recombinant protein purification typically involve some form of column-based separation and, as such, require resins that are both expensive to manufacture and to purchase, and are only suitable for small-scale laboratory operations due to the inherent nature of column based separations, giving rise to significant protein loss and the recovery of only small quantities. Additionally, current methods for larger scale purifications must be achieved by a series of buffered extractions and centrifuging. Such large scale processes can be solvent intensive and do not necessarily give rise to pure products or significant yields. Methods for large-scale, efficient purification of selected proteins are essential to the pharmaceutical industries as well as other biochemistry-based concerns.

A need thus exists for an efficient method for purification of recombinant proteins. Ideally, such a purification medium would be self-supported, inexpensive to manufacture, and as effective as existing methods. In addition, it would be desirable to have a purification medium that is easily recovered and could be configured for use in continuous processes in any desired structure. Finally, it would be desirable if such a medium could also be used in environmental remediation of heavy metals.

### SUMMARY OF THE INVENTION

Various features and advantages of the invention will be set forth in part in the following description, or may be obvious from the description, or may be learned through practice of the invention.

Generally, in one embodiment, the present invention is directed to a self-supported purification medium for recombinant protein purification and to a process for using that medium. A self-supported purification medium does not require a column or special machinery and, therefore, is inexpensive to manufacture while retaining the effectiveness of existing methods. In addition, a self-supported purification medium is easily recovered and can be configured for use in continuous processes in any desired structure. The purification medium may comprise a polymer substrate, a chelating agent that is formed from the reaction of the substrate with a dianhydride precursor, and a transition metal ion having a 2+ oxidation state. The polymer substrate may contain amine groups or hydroxy groups. The amine groups or hydroxy groups of the polymer substrate are reacted with the dianhydride precursor to form the chelating groups that are covalently bonded to the substrate. The chelating substrate is then complexed with the transition metal ion having a 2+ oxidation state. The self-supported purification medium is next contacted with a composition containing a histidine-tagged protein. The histidine-tagged protein binds with the chelated transition metal ion having a 2+ oxidation state thereby separating the histidine-tagged protein from the composition.

In certain embodiments, the chelating agent precursor may comprise EDTA, dianhydride (ethylenediaminetetraacetic dianhydride). In other embodiments, the chelating agent precursor may comprise DTPA, dianhydride (diethylenetriaminepentaacetic dianhydride). In certain embodiments, the polymer substrate may be chosen from a group consisting of PVOH (polyvinyl alcohol), cellulose, a derivative of cellulose, a polyester, Tencel® (Tencel®) is the brand name of Tencel® Ltd. for lyocell, the fiber's generic name), nylon, wool (keratin) or cotton. Also, in certain embodiments, the polymer substrate may be a fiber while in other embodiments the polymer substrate may be a film. In certain embodiments the fiber may have a diameter of less than 100 microns and may be considered a standard fiber or micro fiber while in other embodiments the fiber may have a diameter of less than 1 micron and will be considered a nano fiber. In certain embodiments the polymer substrate may be a non-woven, woven, knitted fabric, film or a polymer nanoparticle. The polymer substrate may be porous, fibrillated, etched or treated in such a way as to give rise to an enhanced surface area.

In preferential embodiments of the present invention, the transition metal ion may be chosen from Co²⁺ or Ni²⁺. In other embodiments, the transition metal ion may be chosen from Fe²⁺, Cu²⁺, and Zn²⁺. Still in further embodiments, any transition metal with affinity for the chelating substrate may be used.

In certain embodiments of the present invention, the process for purifying recombinant proteins may further comprise the step of contacting the self-supported purification medium bound to the histidine-tagged protein with a washing composition capable of removing the histidine-tagged protein from the self-supported purification medium. In certain embodiments, the washing composition may contain an imidazole.

Also, in certain embodiments of the present invention, the process for purifying recombinant proteins may further comprise the step of filtering the washing composition in order to separate the histidine-tagged protein from the washing composition.

In another exemplary embodiment of the present invention, self-supported filter media comprising a polymeric substrate is taught. The polymeric substrate comprises a polymeric material containing amine groups or hydroxy groups that have been reacted with and covalently bonded to a chelating agent precursor such as EDTA, dianhydride or DTPA, dianhydride. The chelating agent is then complexed with a transition metal ion having any oxidation state. The transition metal ion is inherently configured to bind to chelating species for separately self-supported filtering of the species from a composition contacted with the chelating polymeric substrate.

In still another exemplary embodiment of the present invention, a process for purifying recombinant proteins is provided for. The process involves providing a purification medium. The purification medium is made up of a polymer substrate containing amine groups and/or hydroxy groups, reacted with EDTA, dianhydride (or DTPA dianhydride), which is then chelated to a transition metal ion having a 2+ oxidation state. The amine groups or hydroxy groups of the polymer substrate are covalently bonded to the EDTA, dianhydride through the reaction of these groups with the anhydride groups of the reagent. The EDTA, dianhydride is then complexed with the transition metal ion having a 2+ oxidation state. The purification medium is next contacted with a composition containing a histidine-tagged protein. The histidine-tagged protein binds with the chelated transition metal ion having a 2+ oxidation state thereby separating the histidine-tagged protein from the composition.

In yet another embodiment, a process for removal of transition metals is provided. The process involves providing a chelating structure made up of a polymer substrate. The polymer substrate is covalently bonded to EDTA, dianhydride (or DTPA dianhydride). The polymer substrate is made up of a polymer containing amine groups or hydroxy groups that have reacted with the EDTA, dianhydride. The chelating structure is brought into contact with a transition metal ion in liquid solution. The EDTA, dianhydride becomes complexed with the transition metal ion. The resultant composition can then be disposed of by landfill or by incineration or the transition metals can be recovered by adjustment of pH in solution. The pH adjustment is dependent on the particular transition metal being recovered. Additionally, the adjustment of pH could provide a basis for the selective recovery of transition metals from a mixture.

### BRIEF DESCRIPTION OF THE FIGURES

A full and enabling disclosure of the present invention, including the best mode thereof, to one of ordinary skill in the art, is set forth more particularly in the remainder of the specification, including reference to the accompanying figures, in which:

FIG. 1 illustrates purification using the chelating fiber assembly of the present invention;

FIG. 2 illustrates the cobalt contents of PVOH fibers treated with EDTA, dianhydride over varying amounts of time;

FIG. 3 illustrates the cobalt contents of Tencel® fibers treated with varying amounts of EDTA, dianhydride;

FIG. 4 illustrates a comparison of a Tencel® wet-laid sheet and a non-woven of the same mass;

FIG. 5 illustrates cobalt contents of Tencel® fibers treated with varying amounts of EDTA, dianhydride;

FIG. 6 illustrates cobalt contents of cotton fibers treated with varying amounts of EDTA, dianhydride.

### DETAILED DESCRIPTION OF REPRESENTATIVE EMBODIMENTS

Reference now will be made to the embodiments of the invention, examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

In general, the present invention is directed to a chelating composition which may be used in a self-supported purification medium for recombinant protein purification. In addition, owing to economies of scale, the material may also be used in environmental remediation of heavy metals.

When used as a purification medium, the composition may comprise a polymer substrate that is reacted with a chelating agent precursor to form a covalently bonded chelating agent with the chelating agent then being complexed with a transition metal ion, such as a metal ion having a 2+ oxidation state. Histidine-tagged proteins possess a well known pH dependent affinity for transition metal ions having a 2+ oxidation state. Transition metal ions having oxidation states of 1+, 3+, or 4+may also be utilized though a 2+ oxidation state is preferred. In one exemplary embodiment, a fiber-based substrate for histidine-tagged recombinant protein purification is taught. This method is designed to be effective based on the established pH dependent affinity of histidine-tagged proteins for certain metal ions. By utilizing a novel method for chelating metal ions onto fiber surfaces, histidine-tagged proteins can be purified in a continuous manner without the use of column-based separation. In some embodiments, the present invention may also be utilized for removal of certain metal ions from effluent. The separations are usually performed at ambient temperatures. If the separation is specific to protein separation, the temperature must not exceed the temperature at which the protein would be denatured and rendered useless.

As previously stated, the self-supported purification medium generally contains a polymer substrate covalently bound to a chelating agent that is then complexed to a transition metal. Some examples of suitable polymer substrates are PVOH, Tencel®, cellulose, a derivative of cellulose, polyester, nylon, wool, cotton, keratin or any other substrate that would be readily recognized by persons of ordinary skill in the art. In general, the polymers used contain hydroxyl and amine functional groups that have the ability to react with an acyl anhydride (organic acid anhydride). In some exemplary embodiments, common, inexpensive materials may be utilized to make a chelating fiber.

A variety of suitable polymer substrates are available such as textiles, woven fabrics, non-woven webs, films, etc. In one exemplary embodiment the polymer substrate is a non-woven web, which is available in many forms and can be easily manufactured by those skilled in the art. The effectiveness of these webs is dependent on the fiber size, shape, porosity and density of the non-woven. Fibers commonly used in the art to manufacture non-woven webs, such as pulp fibers, natural and synthetic fibers, thermomechanical pulp fibers, or mixtures thereof, can be utilized. In addition, the fibers used in making the non-woven web may have any suitable morphology and may include hollow or solid, porous, straight or crimped, capillary, electrospun, single component, conjugate or multi-component fibers or filaments, and blends or mixtures of such fibers, as are well known in the art. In some exemplary embodiments, suitable fibers have a diameter ranging from 20 nanometers to 100 microns. In some embodiments, fibers with a high surface area are utilized.

In addition, the self-supported purification medium of the present invention contains a chelating agent covalently bonded to the polymer substrate. A chelating agent is known to persons of ordinary skill in the art as an organic compound capable of forming coordinate bonds with metals through two or more atoms of the organic compound. Suitable chelating agent precursors include EDTA, dianhydride (ethylenediaminetetraacetic dianhydride) and DTPA, dianhydride (diethylenetriaminepentaacetic dianhydride) or any other substance, typically an acyl anhydride (organic acid anhydride) capable of reaction with hydroxyl or amino groups, that would be readily recognized by persons of ordinary skill in the art. In the present invention, the chelating agent is formed from the reaction of a dianhydride with the reactive groups of a polymer substrate which gives rise to the provision of chelating species covalently bonded to the substrate.

In some exemplary embodiments, a fibrous polymer substrate is treated with EDTA, dianhydride in an appropriate medium to give rise to the formation of covalent bonds between the amine or hydroxy groups of the polymer substrate and the opened anhydride rings of EDTA, dianhydride. This reaction forms a chelating species on the polymer substrate known as ethylenediaminediaceticacid, or the EDDA ligand. This EDDA ligand comprises four chelating species comprising two tertiary amine groups and two carboxylic acid groups while the ligand is attached to the substrate via two ester linkages (or amide linkages if the polymer contains amino rather then hydroxyl groups) that were formerly part of each of the anhydride groups in the EDTA, dianhydride. In embodiments where a PVOH polymer substrate is utilized, it is desirable to wash the PVOH in a solvent such as toluene (since PVOH is soluble in DMF and not in toluene) or any suitable solvent known to one of ordinary skill in the art and then cold distilled water, following the reaction of the EDTA, dianhydride with the substrate. In other embodiments, after the fiber polymer substrate has reacted with EDTA, dianhydride, it is washed in a hot solvent such as DMF and then distilled water.

As stated, the chelating EDDA ligand is complexed with a transition metal ion, such as a metal ion having a 2+ oxidation state. Suitable transition metals include Fe²⁺, Cu²⁺, Zn²⁺, Co²⁺, Ni²⁺ or any other transition metal that would be readily recognized by one of ordinary skill in the art. In some exemplary embodiments, the EDDA ligand has two amine groups and two carboxylic acid groups that form coordinate bonds with Co²⁺. The self-supported purification medium of the present invention generally contains a polymer substrate covalently bound to a chelating ligand that is complexed to a transition metal.

In accordance with the present invention, the self-supported purification medium is utilized for recombinant protein purification. In some exemplary embodiments, histidine-tagged proteins can be engineered, expressed and contained in a suitable liquid medium. The self-supported purification medium is contacted with a composition containing a histidine-tagged protein. In some embodiments, a neutral pH is desired in order to attach the histidine-tagged protein to the transition metal. The histidine-tagged protein binds with the transition metal ion and separates from the composition. The histidine-tag allows the recombinant protein to bind to a transition metal in a cooperative manner so that it forms a strong complex. Such a complex allows for essentially no background metal leakage from the complex so that the purified recombinant protein is not contaminated with undesired protein from the mixture being purified. In some embodiments, where the substrate is used to recover transition metals from media, the cobalt or other transition metal is removed by adjusting the pH so that the metal is recovered and the substrate reused.

In yet another embodiment, a process for removal of transition metals is provided. The process involves providing a chelating structure made up of a polymer substrate. The polymer substrate is covalently bonded to EDTA, dianhydride. The polymer substrate is made up of a polymer containing amine groups or hydroxy groups that have reacted with the EDTA, dianhydride. The chelating structure is brought into contact with a transition metal ion in liquid solution. The EDTA, dianhydride becomes complexed with the transition metal ion. The resultant composition can then be disposed of by landfill or by incineration or the transition metals recovered by adjustment of pH in solution. The pH of treatment is dependent on the particular transition metal being recovered. Additionally, the adjustment of pH could provide a basis for the selective recovery of transition metals from a mixture.

The present invention may be better understood with reference to the following examples:

### EXAMPLE 1

Poly(Vinyl Alcohol) Non-Woven as Substrate for Chelating Fiber Manufacture.

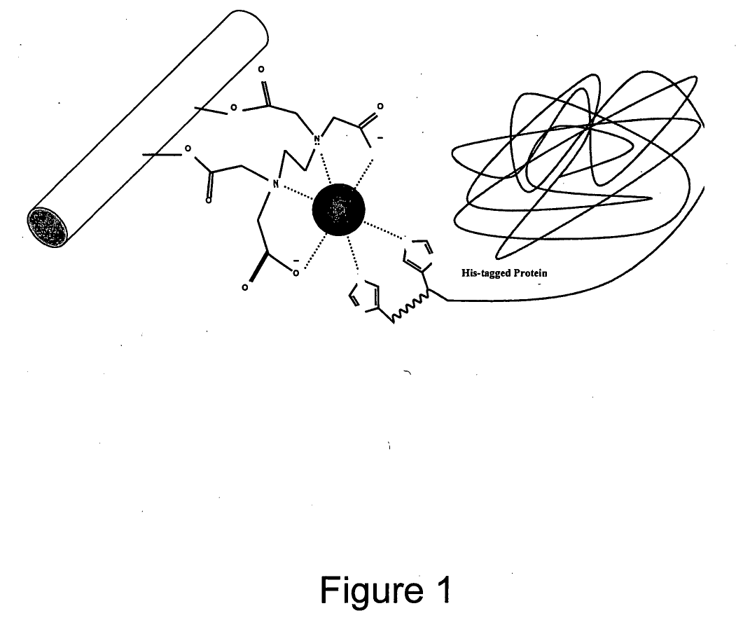

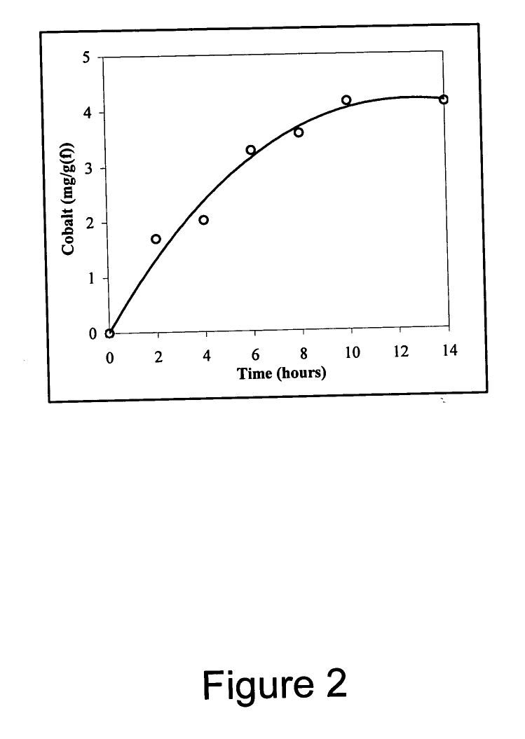

PVOH (1 g) in toluene (100 ml) is refluxed (111° C.) for 2, 4, 6, 8, 10 or 14 hours with 1 g of EDTA, dianhydride. The substrate is removed and refluxed in hot toluene for 15 minutes and washed twice in cold water for 15 minutes and finally cold ethanol for 15 minutes and dried. The modified fiber is then treated in a solution of Cobalt (II) Chloride for 15 minutes, washed twice in cold deionised water and dried. The chelating fiber product is then used to purify a recombinant protein (FIG. 1). Analysis of the cobalt contents of the samples showed the contents to be 0, 1.69, 2.02, 3.28, 3.57, 4.14, and 4.12 mg (Co)/g (fiber) with respect to the reaction time. (FIG. 2)

### EXAMPLE 2

1.5 Denier Tencel® Non-Woven as Substrate for Chelating Fiber Manufacture.

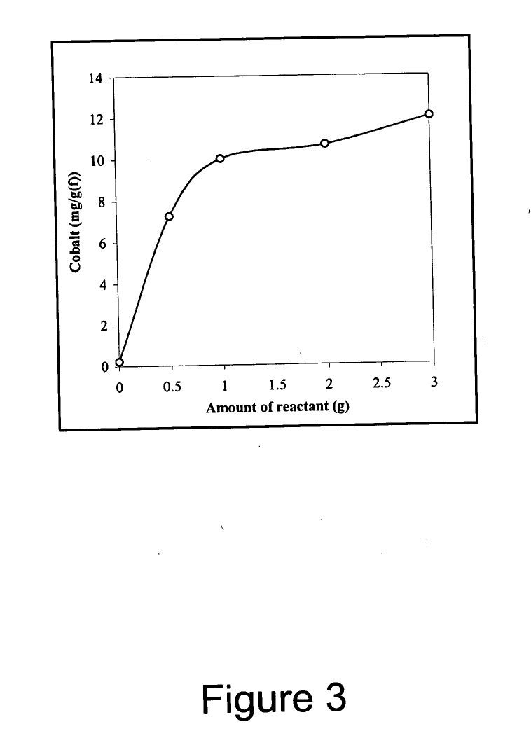

Tencel® (2 g) in DMF (100 ml) is refluxed (153° C.) with 0, 0.5, 1, 2 or 3 g of EDTA, dianhydride for 2 hours. The substrate is removed and refluxed in hot DMF for 15 minutes and washed twice in cold water for 15 minutes and dried. The modified fiber is then treated in a solution of Cobalt (II) Chloride for 15 minutes, washed twice in cold deionised water and dried. The chelating fiber product is then used to purify a recombinant protein. Analysis of the cobalt contents of the samples showed the contents to be 0.22, 7.26, 10.02, 10.67 and 12.02 mg (Co)/g (fiber) with respect to the amount of EDTA, dianhydride used. (FIG. 3)

### EXAMPLE 3

Tencel® Wet-Laid Sheet as Substrate for Chelating Fiber Manufacture.

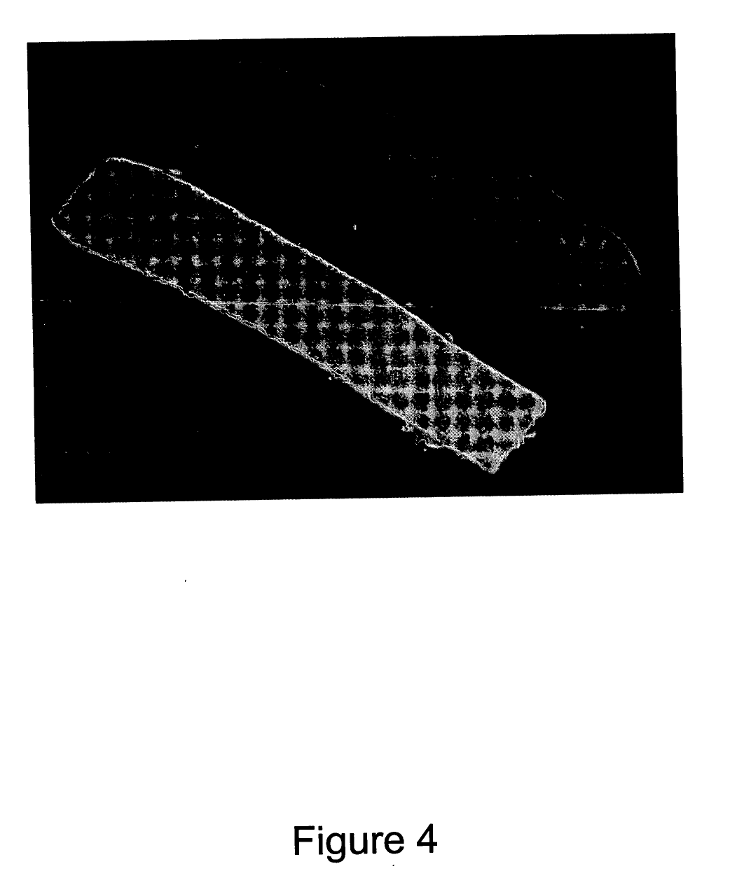

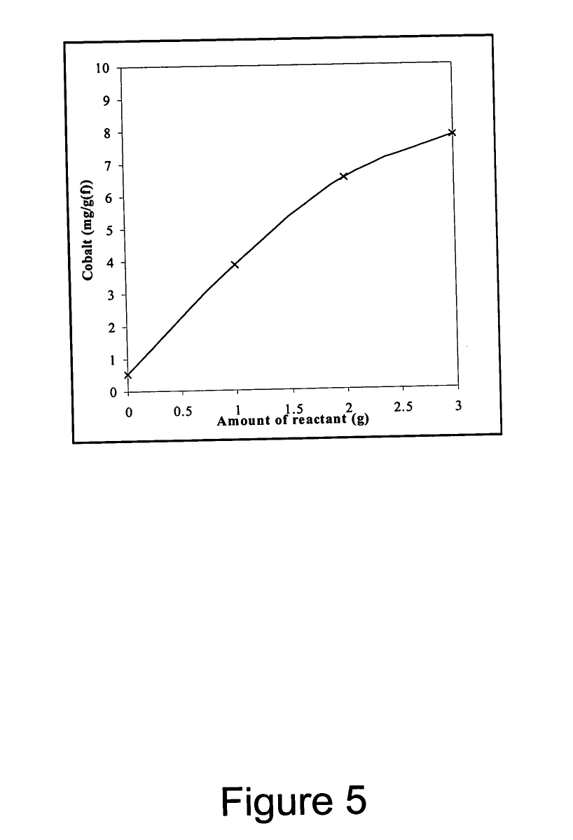

Tencel® (2 g) in DMF (100 ml) is refluxed (153° C.) with 0, 1, 2 or 3 g of EDTA, dianhydride for 2 hours. The substrate is removed and refluxed in hot DMF for 15 minutes and washed twice in cold water for 15 minutes and dried. The modified fiber is then treated in a solution of Cobalt (II) Chloride for 15 minutes, washed twice in cold deionised water and dried. The chelating fiber product (FIG. 4) is then used to purify a recombinant protein. Analysis of the cobalt contents of the samples showed the contents to be 0.49, 3.37, 4.93, 6.58 mg (Co)/g (fiber) with respect to the amount of EDTA, dianhydride used. (FIG. 5)

### EXAMPLE 4

2.7 Micronaire Cotton Non-Woven as Substrate for Chelating Fiber Manufacture.

Uncatalysed

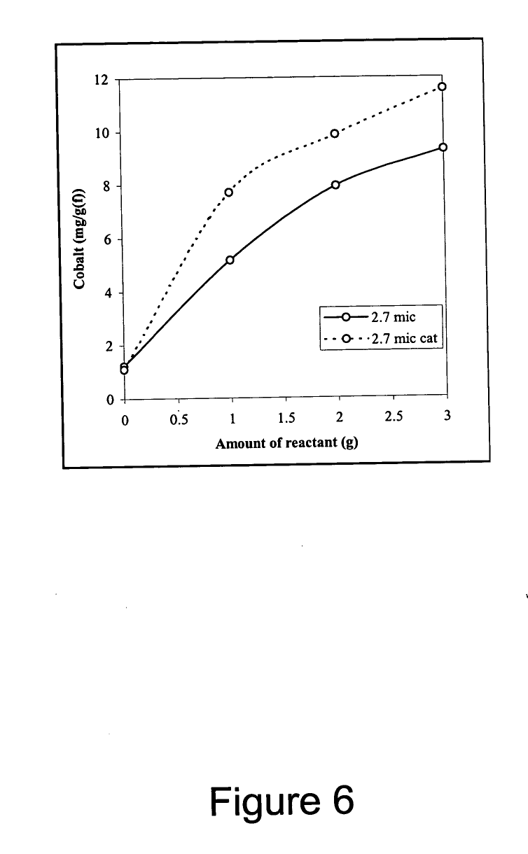

Cotton (2 g) in DMF (100 ml) is refluxed (153° C.) with 0, 1, 2 or 3 g of EDTA, dianhydride for 2 hours. The substrate is removed and refluxed in hot DMF for 15 minutes and washed twice in cold water for 15 minutes and dried. The modified fiber is then treated in a solution of Cobalt (II) Chloride for 15 minutes, washed twice in cold deionised water and dried. The chelating fiber product is then used to purify a recombinant protein. Analysis of the cobalt contents of the samples showed the contents to be 1.23, 5.17, 7.93 and 9.29 mg (Co)/g (fiber) with respect to the amount of EDTA, dianhydride used. (FIG. 6)

Catalysed

Cotton (2 g) in DMF (100 ml) is refluxed (153° C.) with 0, 1, 2 or 3 g of EDTA, dianhydride and 20 mg of Scandium (III) trifluoromethanesulfonate (a small amount added as a catalyst of the reaction) for 2 hours. The substrate is removed and refluxed in hot DMF for 15 minutes and washed twice in cold water for 15 minutes and dried. The modified fiber is then treated in a solution of Cobalt (II) Chloride for 15 minutes, washed twice in cold deionised water and dried. The chelating fiber product is then used to purify a recombinant protein. Analysis of the cobalt contents of the samples showed the contents to be 1.11, 7.72, 9.85 and 11.56 mg (Co)/g (fiber) with respect to the amount of EDTA, dianhydride used. (FIG. 6)

1. A process for purifying recombinant proteins comprising:
providing a self-supported purification medium comprising a polymer substrate, said polymer substrate covalently bonded to a chelating ligand, said polymer substrate including a polymer containing amine groups or hydroxy groups that have reacted with a chelating ligand precursor to form the chelating ligand; said chelating ligand being complexed with a transition metal ion having a 2+ oxidation state; and
contacting said self-supported purification medium with a composition containing a histidine-tagged protein, said histidine-tagged protein binding with said transition metal ion thereby separating said histidine-tagged protein from said composition.

2. The process for purifying recombinant proteins of claim 1, wherein said chelating ligand precursor comprises EDTA, dianhydride and said chelating ligand comprises EDDA.

3. The process for purifying recombinant proteins of claim 1, wherein said chelating ligand precursor comprises DTPA, dianhydride and said chelating ligand comprises DTTA.

4. The process for purifying recombinant proteins of claim 1, wherein said polymer substrate is selected from a group consisting of PVOH, a cellulose, a derivative of cellulose, lyocell, a polyester, nylon, wool, cotton, and keratin.

5. The process for purifying recombinant proteins of claim 1, wherein said polymer substrate is a fiber.

6. The process for purifying recombinant proteins of claim 1, wherein said polymer substrate is non-woven.

7. The process for purifying recombinant proteins of claim 1, wherein said polymer substrate is a coating or is particulate.

8. The process for purifying recombinant proteins of claim 1, wherein said polymer substrate is PVOH.

9. The process for purifying recombinant proteins of claim 1, wherein said transition metal ion is selected from the group consisting of Fe²⁺, Cu²⁺, and Zn²⁺.

10. The process for purifying recombinant proteins of claim 1, wherein said transition metal ion is selected from the group consisting of Co²⁺ and Ni²⁺.

11. The process of claim 5, wherein said fiber has a diameter of less than 100 microns.

12. The process of claim 5, wherein said fiber has a diameter of less than 1 micron.

13. The process for purifying recombinant proteins of claim 1, further comprising the step of contacting said self-supported purification medium bound to said histidine-tagged protein with a washing composition capable of removing said histidine-tagged protein from said self-supported purification medium.

14. The process of claim 13, wherein said washing composition comprises an imidazole.

15. The process of claim 13, further comprising the step of filtering said washing composition in order to separate said histidine-tagged protein from said washing composition.

16. A self-supported filter media comprising:
a polymeric substrate comprising a polymeric material containing amine groups or hydroxy groups that have been covalently bonded to a chelating ligand precursor to form a chelating ligand, said chelating ligand being complexed with a transition metal ion having a 2+ oxidation state, said transition metal ion being configured to bind to species for separately self-supported filtering of said species from a composition contacted with said polymeric substrate.

17. The self-supported filter media of claim 16, wherein said chelating ligand precursor comprises EDTA, dianhydride and said chelating ligand comprises EDDA.

18. The self-supported filter media of claim 16, wherein said chelating ligand precursor comprises DTPA, dianhydride and said chelating ligand comprises DTTA (diethylenetriaminetriacetic acid).

19. The self-supported filter media of claim 16, wherein said polymeric substrate is selected from a group consisting of PVOH, a cellulose, a derivative of cellulose, lyocell, a polyester, nylon, wool, cotton, and keratin.

20. The self-supported filter media of claim 16, wherein said polymeric substrate is a fiber.

21. The self-supported filter media of claim 16, wherein said polymeric substrate is non-woven.

22. The self-supported filter media of claim 16, wherein said polymer substrate is a film.

23. The self-supported filter media of claim 16, wherein said polymer substrate is a coating or is particulate.

24. The polymeric substrate of claim 16, wherein said transition metal ion is selected from the group consisting of Fe²⁺, Cu²⁺, and Zn²⁺.

25. The self-supported filter media of claim 16, wherein said transition metal ion is selected from the group consisting of Co²⁺ and Ni²⁺.

26. The polymeric substrate of claim 20, wherein said fiber has a diameter of less than 100 microns.

27. The polymeric substrate of claim 20, wherein said fiber has a diameter of less than 1 micron.

28. The self-supported filter media of claim 16, wherein said negatively charged species is a histidine-tagged protein.

29. A process for purifying recombinant proteins comprising:
providing a purification medium comprising a polymer substrate, said polymer substrate covalently bonded to EDTA, dianhydride, said polymer substrate including a polymer containing amine groups or hydroxy groups that have reacted with said EDTA, dianhydride; said EDTA, dianhydride being complexed with a transition metal ion having a 2+ oxidation state; and
contacting said purification medium with a composition containing a histidine-tagged protein, said histidine-tagged protein binding with said transition metal ion thereby separating said histidine-tagged protein from said composition.

30. The process for purifying recombinant proteins of claim 28, wherein said polymer substrate is selected from a group consisting of PVOH, a cellulose, a derivative of cellulose, lyocell, a polyester, nylon, wool, cotton, and keratin.

31. The process for purifying recombinant proteins of claim 28, wherein said polymer substrate is a fiber.

32. The process for purifying recombinant proteins of claim 28, wherein said polymer substrate is non-woven.

33. The process for purifying recombinant proteins of claim 28, wherein said polymer substrate is a film.

34. The process for purifying recombinant proteins of claim 28, wherein said polymer substrate is a coating or is particulate.

35. The process for purifying recombinant proteins of claim 28, wherein said transition metal ion is selected from the group consisting of Fe²⁺, Cu²⁺, and Zn²⁺.

36. The process for purifying recombinant proteins of claim 28, wherein said transition metal ion is selected from the group consisting of Co²⁺ and Ni²⁺.

37. The process of claim 30, wherein said fiber has a diameter of less than 100 microns.

38. The process of claim 30, wherein said fiber has a diameter of less than 1 micron.

39. The process for purifying recombinant proteins of claim 28, further comprising the step of contacting said purification medium bound to said histidine-tagged protein with a washing composition capable of removing said histidine-tagged protein from said purification medium.

40. The process of claim 38, wherein said washing composition comprises an imidazole.

41. The process of claim 38, further comprising the step of filtering said washing composition in order to separate said histidine-tagged protein from said washing composition.

42. A process for removal of transition metals from an effluent comprising:
providing a chelating structure comprising a polymer substrate, said polymer substrate covalently bonded to EDTA, dianhydride, said polymer substrate including a polymer containing amine groups or hydroxy groups that have reacted with said EDTA, dianhydride; and
contacting said chelating structure with an effluent containing a transition metal ion, said EDTA, dianhydride being complexed with said transition metal ion.

43. The process for removal of transition metals of claim 41, wherein said polymer substrate is selected from a group consisting of PVOH, a cellulose, a derivative of cellulose, lyocell, a polyester, nylon, wool, cotton, and keratin.

44. The process for removal of transition metals of claim 41, wherein said transition metal ion is selected from the group consisting of Fe²⁺, Cu²⁺, and Zn²⁺.

45. The process for removal of transition metals of claim 41, wherein said transition metal ion is selected from the group consisting of Co²⁺ and Ni²⁺.

46. The process for removal of transition metals of claim 41, wherein said transition metal ion is selected from the group consisting all transition metals.
